(19) United States
(12) Patent Application Publication
(10) Pub. No.: US 2019/0240410 A1
(43) Pub. Date: Aug. 8, 2019

(54) IMPROVEMENTS IN OR RELATING TO TRANSDERMAL DELIVERY

(71) Applicant: NDM TECHNOLOGIES LIMITED, Leicestershire (GB)

(72) Inventors: Dewan Fazlul Hoque CHOWDHURY, Leicestershire (GB); Paul BURNETT, Foston (GB)

(73) Assignee: NDM TECHNOLOGIES LIMITED, Leicestershire (GB)

(21) Appl. No.: 16/319,042

(22) PCT Filed: Jul. 12, 2017
(86) PCT No.: PCT/GB2017/052045

§ 371 Date: Jan. 18, 2019

(30) Foreign Application Priority Data
Jul. 19, 2016 (GB) 1612491.9

(51) Int. Cl. A61M 5/24; A61M 5/142; A61M 5/20

(52) U.S. Cl. CPC A61M 5/24; A61M 5/14244; A61M 2005/1585; A61M 2005/14252; A61M 5/20

(57) ABSTRACT

It is known to deliver drugs through the skin, i.e. transdermally, by using a needle that is used in conjunction with a device. A device (10), for delivering a needle (110) to an injection site, comprises a rotation member (12) which is rotatable about a reference axis (A_{ref}), the reference axis (A_{ref}) being the central axis of the rotation member (12). The device (10) further including a needle support member (14) which is configured to support a needle (110) in use. The needle support member (14) being fixedly secured to the rotation member (12) such that rotation of the rotation member (12) results in the needle (110) supported by the needle support member (14) in use being delivered to an injection site. The central axis (A_{OffSet}) of the needle support member (14) is parallel to the reference axis (A_{ref}) such that the needle support member (14) is offset from the rotation member (12).

This invention relates to a device for delivering a needle to an injection site.

It is known to deliver drugs through the skin, i.e. transdermally, by using a needle.

When penetrating a needle into the skin for drug administration or diagnostics purposes, whether for animal or human applications, it can be advantageous to position the needle relative to the skin at an angle other than 90°. A preferable angle of penetration is 70°. Penetrating a needle into the skin at such an angle allows the needle to slice or cut through the skin, which makes the penetration more reliable, while not causing any more trauma to the skin than when a needle is penetrated at 90°.

Moreover, some needles (particularly microneedles) have a tendency to rebound off the skin when they are applied perpendicular to the skin, thus preventing them from penetrating the skin. This can also cause the needles to buckle which can often lead to the needle snapping.

The word device is used herein to describe a system or structure into which a needle is loaded. The word needle is used here to describe one or more needles and associated housing and conduits where present. The term vial is used to describe a reservoir for the storage of a drug or other pharmaceutical agent, biological drug, vaccine, or cosmetic agent, intended for administration into a patient who may be a human or animal subject.

According to an aspect of the invention there is provided a device, for delivering a needle to an injection site, comprising:
a rotation member rotatable about a reference axis, the reference axis being the central axis of the rotation member; and
a needle support member configured to support a needle in use, the needle support member being fixedly secured to the rotation member such that rotation of the rotation member results in the needle supported by the needle support member in use being delivered to an injection site,
wherein the central axis of the needle support member is parallel to the reference axis such that the needle support member is offset from the rotation member,
wherein the support member permits a needle, in use, to be delivered to the injection site along an arcing pathway.

The above-described arrangement of the rotation member and the needle support member permits a needle, in use, to be delivered to an injection site along an arcing pathway. Moreover, the central axis of the needle support member being parallel to the reference axis means that the needle support member is offset from the reference axis, which results in the needle penetrating the skin at an angle (i.e. a penetration angle) other than 90°.

The reference axis being the central axis of the rotation member means that if the rotation member moves laterally, the reference axis also moves laterally as it is always the central axis of the rotation member.

The distance between the reference axis and the needle support member central axis (i.e. the offset distance) can be chosen so as to provide a particular penetration angle. Preferably, the penetration angle is greater than 0° and less than 90°, such as from 10° to 90°, for example from 30° to 80°, ideally from 50° to 70°. More preferably, the penetration angle is about 70°.

The aforementioned advantages of the device are achieved whilst maintaining a compact device which is flat when administering the needle. For example, providing a rotation member with a large diameter may change the penetration angle of the needle, but would result in a cumbersome device.

Moreover such device can be used in conjunction with different types of needles and for delivering at different depths of the skin whilst still providing a flat and compact device. For example, the device might be used with a microneedle to deliver at a depth of a 1.5 mm, or instead might be used with a flexible needle to deliver at a depth of about 40 mm.

In a particular embodiment of the invention, the distance between the reference axis and the needle support member central axis is between 2 and 3 mm and is preferably 2 mm.

Optionally the device further includes a vibration member configured to vibrate relative to the device. The vibration member is preferably configured to vibrate prior and/or during rotation of the rotation member. The vibration member may be positioned relative to the needle support member so as to vibrate around the injection site.

The vibration member provides vibrations to the skin which can relax the muscles under the skin. Relaxing the muscles is advantageous prior to and/or during the penetration of a needle so as to make the penetration easier and less painful for a patient. This is particularly true when the penetration is to be into the muscle, i.e. an intramuscular injection.

The vibration member may be a mechanical component such as a coiled spring which is released so as to cause a vibration.

Optionally the device further includes an elongate needle sleeve extending from the needle support member, the needle sleeve being configured to receive a needle in use.

The inclusion of such a needle sleeve provides structural support for the needle when the device is in use. The needle sleeve may extend along at least half of or the majority of the length of the needle so as to prevent the needle from buckling when it contacts the skin. This is especially advantageous when the needle is long such as those used for intramuscular injections.

The needle sleeve may extend continuously around the circumference of the needle or it may instead extend non-continuously around the circumference of the needle (i.e. it may be made from more than one needle sleeve portion).

Preferably the device further includes an actuator surface in contact with the rotation member, wherein lateral movement of the actuator surface causes the rotation member to rotate.

The inclusion of such an actuator surface provides a means for rotating the rotation member so as to provide a needle at the injection site without having to necessarily physically move the device relative to the injection site.

The device may further include an actuator configured to laterally move the actuator surface.

In such embodiments, the rotation member may be rotationally fixed relative to an axis such that it rotates upon movement of the actuator surface but the reference axis (i.e. the central axis of the rotation member) does not laterally move. Alternatively, movement of the actuator surface may cause the rotation member to rotate and laterally move along the actuator surface, such that the reference axis does laterally move.

In an alternative embodiment of the invention the device further includes an actuator configured to directly rotate the rotation member. For example, the actuator may be a motor which directly rotates the rotation member about the reference axis.

In a further embodiment of the invention, the rotation member is manually rotated. For example, the rotation member may be simply rolled over the skin so as to rotate the needle support member and deliver the needle to the penetration site.

Preferably, the rotation member further includes a vial receiving portion configured to receive in use a vial containing a pharmaceutical composition.

Locating the vial receiving portion within the rotation member provides a convenient location for fluid communication to be established between the needle support member and the vial.

According to another aspect of the invention there is provided a needle delivery device comprising:
a device as described hereinabove; and
a needle secured to the needle support member.

Preferably the needle extends substantially perpendicular to the needle support member.

Optionally the needle extends through the needle sleeve. The needle sleeve may extend along the length of the needle.

The needle may be one of the following:
a microneedle;
an intramuscular injection needle; or
a flexible needle.

Preferably the needle includes a needle array comprising more than one needle.

According to a further aspect of the invention there is provided a needle delivery device assembly comprising:
a needle delivery device as described hereinabove; and
a vial containing a pharmaceutical composition.

The vial may be housed within the rotation member.

According to a further aspect of the invention there is provided a method of administering a drug to a patient using a needle delivery device assembly as described hereinabove.

There now follows a brief description of preferred embodiments of the invention, by way of non-limiting examples, with reference being made to the accompanying drawings in which:

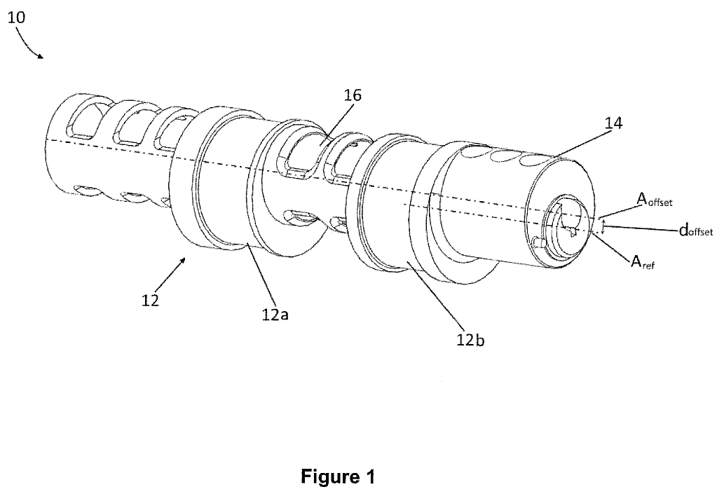

FIG. 1 shows a perspective view of a device according to an embodiment of the invention;

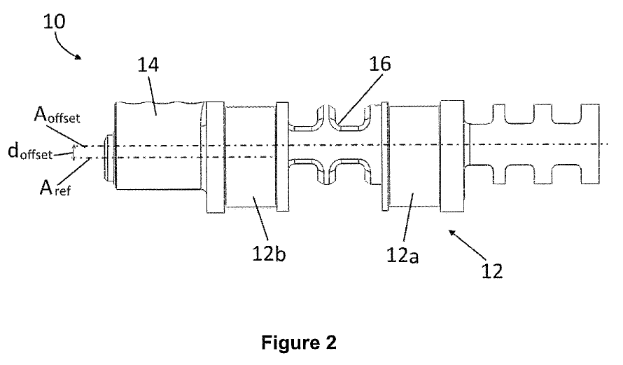

FIG. 2 shows a side elevation view of the device shown in FIG. 1;

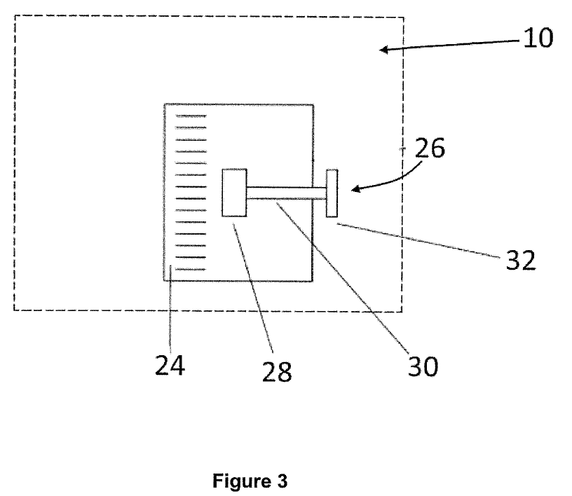

FIG. 3 shows a schematic plan view of an actuator surface and actuator of a device according to another embodiment of the invention; and

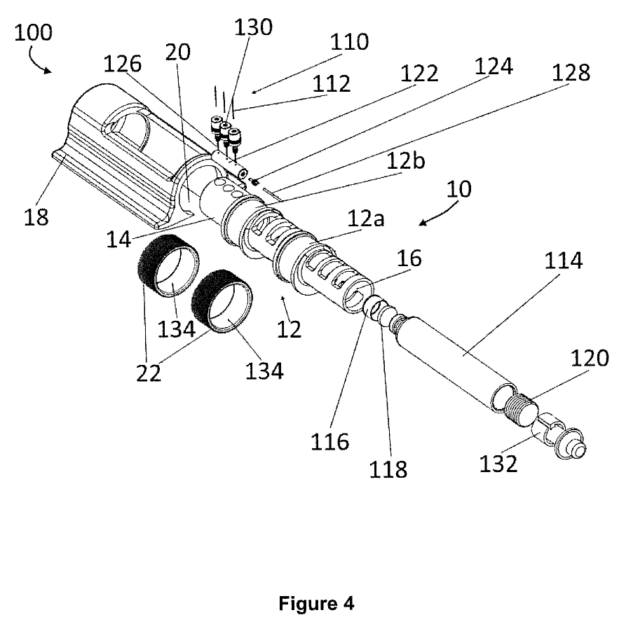

FIG. 4 shows an exploded view of a needle delivery device according to a further embodiment of the invention.

A device according to a first embodiment of the invention is shown in FIGS. 1 to 3 and is designated generally by reference numeral 10.

The device 10 includes a rotation member 12 which is rotatable about a reference axis A_{ref}, the reference axis being the central axis of the rotation member 12. In the embodiment shown, there are two rotation members portions 12a, 12b, but in other embodiments there may be fewer or more rotation member portions.

The device 10 also includes a needle support member 14 which is configured to support a needle in use. The needle support member 14 might be configured to house the needle and/or to hold the needle in position relative to the needle support member 14.

The needle support member 14 is fixedly secured to the rotation member 12 such that when the rotation member 12 is rotated in use it results in the needle being supported by the needle support member 14 being delivered to an injection site.

The central axis A_{offset} of the needle support member 14 (i.e. the axis that runs along the length of the needle support member 14 via its centre) is parallel to the reference axis A_{ref}.

In particular, the distance d_{offset} between the reference axis A_{ref} and the needle support member central axis A_{offset} in the embodiment shown is 2 mm. In other words, the needle support member 14 is centrally offset from the rotation member 12 by 2 mm. In other embodiments of the invention, the offset distance may be another value.

In the embodiment shown, the rotation member 12 has a largest diameter of 20 mm. It will be appreciated that in other embodiments of the invention the rotation member 12 may have a different diameter. For example, the rotation member may have a diameter of between 16 and 24 mm. In another example, where the device 10 is configured to be used with a 0.5 ml capacity vial, the diameter may be 5 mm.

The device 10 also includes a vibration member (not shown) which is configured to vibrate prior and/or during rotation of the rotation member 12 (i.e. before and/or during delivery of the needle to the injection site). The vibration member causes the skin around the injection site to vibrate.

The device 10 also includes an elongate needle sleeve (not shown) which extends from the needle support member 14 and receives a needle in use.

The rotation member 12 further includes a vial receiving portion 16 which houses a vial containing a pharmaceutical composition.

The device 10 also includes a housing 18 (not shown in FIG. 1 or 2 but is shown in FIG. 4) which houses the rotation member 12 and the needle support member 14, and which includes an opening 20 through which a portion of the rotation member 12 protrudes.

As also shown in FIG. 4, the device may include a rubber belt 22 surrounding each rotation member portion 12a, 12b. The belt 22 may instead be made from another material.

In use, the rotation member 12 is manually rolled along a patient's skin so as to rotate the needle support member 14 and thus deliver a needle to an injection site.

In an alternative embodiment of the invention, an actuator, such as a motor or a mechanically wound coil, may instead rotate the rotation member 12 such that the device 10 does not have to be rolled along the skin.

In a further embodiment of the invention the device 10 may also include an actuator surface 24 which is in contact with the rotation member 12. FIG. 3 schematically shows such an actuator surface (the rotation member 12 is not shown in FIG. 3 for clarity purposes).

This embodiment also includes an actuator assembly 26 which is configured to laterally move the actuator surface 24.

The actuator assembly 26 includes an actuator 28 which is operatively coupled to an interface 30 which is in turn operatively coupled to a control mechanism 32. The control mechanism 32 may be a mechanically wound coil which when released will turn the interface 30 (in the form of a drive shaft, for example). Turning of the interface 30 will turn the actuator 28, which may be a cog, and thus will cause the lateral movement of the actuator surface 24.

Lateral movement of the actuator surface 24 will then cause lateral movement and rotation of the rotation member 12 and thus the needle support member 14.

The actuator surface 24 may be a track and the rotation member 12 may also include a tracked surface.

The actuator surface 24 may be moved by other means which would be apparent to the person skilled in the art.

A needle delivery device according to another embodiment of the invention is shown in FIG. 4 and is designated generally by reference numeral 100.

The needle delivery device 100 includes the device 10 as described hereinabove and a needle array 110 secured to the needle support member 14. The needle array 110 includes three hypodermic microneedles 112, but may instead include fewer or more microneedles 112.

The needle delivery device 100 may instead include a single needle, and the needle or needles may take another form such as an intramuscular injection needle and/or a flexible needle.

The needles 112 may be solid metal, plastic, ceramic or glass.

The needle delivery device 100 further includes the following features:
114—3 ml insulin vial
116—insulin vial cap
118—insulin vial septum
120—insulin rubber septum
122—needle thimble
124—needle adhesive
126—needle adhesive
128—hypodermic needle
130—needle hub
132—plastic plunger
134—roller belt adhesive

It will be appreciated that the mechanisms discussed above have been discussed in the context of manual operation, but could all be equally automated using appropriate electro-mechanical actuation means.

It will also be appreciated that the needle delivery device 100 may be used to deliver a pharmaceutical composition other than insulin and may contain a vial having a different volume than set out above.

1. A device, for delivering a needle to an injection site, comprising:
a rotation member rotatable about a reference axis, the reference axis being the central axis of the rotation member; and
a needle support member configured to support a needle in use, the needle support member being fixedly secured to the rotation member such that rotation of the rotation member results in the needle supported by the needle support member in use being delivered to an injection site,
wherein the central axis of the needle support member is parallel to the reference axis such that the needle support member is offset relative to the rotation member,
wherein the support member permits a needle, in use, to be delivered to the injection site along an arcing pathway.

2. The device according to claim 1 wherein the distance between the reference axis and the needle support member central axis is between 2 and 3 mm.

3. The device according to claim 1 or 2 wherein the distance between the reference axis and the needle support member central axis is 2 mm.

4. The device according to any preceding claim further including a vibration member configured to vibrate relative to the device.

5. The device according to claim 4 wherein the vibration member is configured to vibrate prior and/or during rotation of the rotation member.

6. The device according to claim 4 or 5 wherein the vibration member is positioned relative to the needle support member so as to vibrate around the injection site.

7. The device according to any preceding claim further including an elongate needle sleeve extending from the needle support member, the needle sleeve being configured to receive a needle in use.

8. The device according to any preceding claim further including an actuator surface in contact with the rotation member, wherein lateral movement of the actuator surface causes the rotation member to rotate.

9. The device according to claim 8 further including an actuator configured to laterally move the actuator surface.

10. The device according to any one of claims 1 to 7 further including an actuator configured to directly rotate the rotation member.

11. The device according to any preceding claim wherein the rotation member further includes a vial receiving portion configured to receive in use a vial containing a pharmaceutical composition.

12. A needle delivery device comprising:
a device according to any one of claims 1 to 11; and
a needle secured to the needle support member.

13. The needle delivery device according to claim 12 wherein the needle extends substantially perpendicular to the needle support member.

14. The needle delivery device according to claim 12 or claim 13 when dependent on claim 7 wherein the needle extends through the needle sleeve.

15. The needle delivery device according to any one of claims 12 to 14 wherein the needle is one of the following:
a microneedle;
an intramuscular injection needle; or
a flexible needle.

16. The needle delivery device according to any one of claims 12 to 15 wherein the needle includes a needle array comprising more than one needle.

17. A needle delivery device assembly comprising:
a needle delivery device according to any one of claims 12 to 16; and
a vial containing a pharmaceutical composition.

18. A method of administering a drug to a patient using a needle delivery device assembly according to claim 17.

19. A device generally as herein described with reference to and/or as illustrated in FIGS. 1 to 3.

20. A needle delivery device assembly as herein described with reference to and/or as illustrated in FIG. 4.
